(19) United States
(12) Patent Application Publication
(10) Pub. No.: US 2024/0268983 A1
(43) Pub. Date: Aug. 15, 2024

(54) THORACIC ORTHOSIS

(71) Applicant: ax-lightness composites GmbH, Duderstadt (DE)

(72) Inventors: Bernd Nolte, Duderstadt (DE); Klaus Lotz, Aßlar (DE)

(21) Appl. No.: 18/569,316

(22) PCT Filed: Jun. 27, 2022
(86) PCT No.: PCT/EP2022/067617

§ 371 Date: Dec. 12, 2023

(30) Foreign Application Priority Data
Jul. 2, 2021 (DE) 10 2021 117 132.6

(51) Int. Cl. A61F 5/02

(52) U.S. Cl. CPC A61F 5/026

(57) ABSTRACT

A thoracic orthosis (10) is provided for relieving a human user's spine when required. The thoracic orthosis has a back rail (12) resting, when in use, with a ventral flat side against back extensor muscles adjacent to both sides of the user's spine in the region of the thoracic and at least the upper lumbar vertebrae of the user. A shoulder carrier system is connected to the back rail (12) and has two carrier elements (14) reaching around the user's shoulders in a backpack-style when in use. The back rail (12) is made in one piece from a sheet-like carbon material that is compression-resistant in its direction of longitudinal extension and is sagittally bending-elastic.

Field of the invention. The invention relates to a thoracic orthosis for relieving a human user's spine when required.

State of the art. A thorax orthosis of this type is known from DE 10 2011 076 843 B4 discloses a thorax orthosis that comprises a back rail resting, when in use, with a ventral flat side against back extensor muscles adjacent to both sides of the user's spine in the region of the thoracic and at least the upper lumbar vertebrae of the user, and a shoulder carrier system connected to the back rail and having two carrier elements reaching around the user's shoulders in a backpack-style when in use.

In many situations in everyday life, the human spine is exposed to considerable strain. For example, when lifting and carrying heavy loads in front of the body, such as when transporting bricks, cement or similar in the construction industry. But also, when transporting loads shouldered like backpacks, such as oxygen cylinders in fire department rescue operations. The human spine is also temporarily exposed to considerable loads in many sports, such as equestrian sports, especially show jumping. In an optimal sequence of movements, all these loads are essentially compression loads in the longitudinal direction of the spine, i.e. cranial-caudal, which are partially absorbed by the natural S-curvature of the spine in the medial sagittal plane, whereby the external forces are transferred via the spine into the adjacent muscles and ultimately dissipated there. However, the respective movement can often not be optimally coordinated, so that bending, torsion and shearing movements can also occur. Excessively high or excessively long loads can have adverse health consequences. This is all the more the case the more the respective forces act laterally and the more difficult it is for the natural S-curvature of the spine to absorb them. A number of orthoses are therefore known to protect the spine from overloading. Their common task is to divert the external forces away from the spine to less sensitive skeletal structures of the body. The term orthosis is used here as a collective term for body-hugging aids that are intended to ensure a balance between mobility and stability of the body. They are used not only after injuries or in the case of limb malpositions, i.e. for medical-therapeutic indications, but also—and in the context of the present invention in particular—without medical indication or objective for purely preventive and supportive stabilization of stressed areas of the body.

An orthosis with a back rail made up of a large number of separate segments is known from DE 10 2011 076 843 B4. The individual segments are connected to the respective neighboring segments via springs. In addition, a tension element is provided and extends longitudinally through all the segments. A belt system consisting of two upper shoulder straps and a lower pelvic belt enables the back rail to be fixed to the user's back in such a way that its individual segments hug against the user's back along the spine, resting centrally on the spine and laterally on the back extensor muscles adjacent to the spine. In the deactivated normal state, the spring elements between the individual segments allow the back rail to follow the user's movements, in particular those of the spine, without significant counterforce. To activate a protective function, a switch can be used to tighten the tension element running through all the segments so that the segments are pressed together to form an essentially rigid bar that is supported between the pelvic belt and the shoulder straps. Forces acting on the shoulders—especially via the arms—such as those that occur when lifting heavy loads, are therefore transferred via the now stiffened back rail past the spine to the pelvic belt and thus to the user's pelvis. In order to allow the user a certain, albeit very limited, freedom of movement even when activated, the individual elements are made of hard-elastic plastic, which allows very limited movement against considerable counterforces.

The known orthosis must be regarded as disadvantageous in several respects. Firstly, the protective function has to be consciously activated or deactivated before or after a load is applied. This is cumbersome and inconvenient for the user and simply unsuitable in all cases in which stress cannot be planned well in advance, such as during rescue operations or in sport. Furthermore, the mechanism described is complicated and requires a lot of space, so that the known orthosis is clearly bulky under or over clothing. In addition, it can hardly be used in combination with loads carried on the back, such as oxygen cylinders, as the weight of the cylinder presses the thick back rail painfully onto the user's spine. And finally, the spine relief of the known orthosis merely consists of shifting the load from one skeletal structure, namely the spine, to another skeletal structure, namely the pelvis, which is only supposedly less sensitive.

A similar orthosis is known from US2015/0133842 A1 and has a very complex system of traction cables and sliders to ensure that the orthosis can be very individually and temporarily adapted to the respective load situation.

DE 20 2010 006 731 U1 discloses an osteoporosis orthosis with a pad in the form of a closed frame made of a carbon material, which is located in a pocket that is fixed to the shoulders, pelvis and abdomen of the wearer by means of a belt system.

EP 1 902 691 B1 also discloses an osteoporosis orthosis with a pad located in a pocket which is fixed to the wearer's shoulders, pelvis and abdomen by means of a belt system. The pad is made of different materials in different vertical areas in order to realize different elasticities there.

US 2013/0261521 A1 discloses a carrying aid that can be individually adjusted to the user's body size and consists of a rigid lower part that embraces the user's pelvis and an equally rigid upper part that rests against the user's shoulders and back, whereby both parts can be moved vertically in relation to each other and can be fixed to each other in various positions.

It is an object of the invention to provide an orthosis for spine relief that can be used variably, is more comfortable for the user to wear and is generally more beneficial to health.

### SUMMARY OF THE INVENTION

This object is achieved by an orthosis for spine relief where the back rail is made in one piece from a sheet-like carbon material that is compression-resistant in its direction of longitudinal extension and is sagittally bending-elastic.

The following comments are provided to ensure an understanding of the terms used in this disclosure. The skilled person will understand that the advantages of the orthosis according to the invention can only be realized in its interaction with the user's body. The spatial and physical characteristics of the orthosis according to the invention can therefore only be described in relation to the user's body, which is expressed here by the reference “when in use” or the like, whereby it should be self-evident that the user's body referred to is not itself part of the invention. Terms such as “ventral”, “dorsal”, “central”, “lateral”, “upper” or “cranial”, “lower” or “caudal” are therefore always to be understood in this sense, i.e. in relation to the upright standing user's body when the orthosis according to the invention is used as intended. The same applies to indications of planes such as “sagittal” or “frontal”, whereby a movement, elasticity or course specified in this way always refers to the movement, elasticity or course in such the according plane.

An aspect of the invention lies in a special choice of material for the orthosis and in particular its back rail. The term “carbon material” here refers to a carbon fiber-reinforced plastic, namely oriented carbon fibers embedded in a duroplastic matrix. In particular, it refers to woven, laid or braided carbon fiber mats embedded in said duroplastic matrix. As is known to the skilled person, it is a particular strength of such carbon materials, as they are known for example as lightweight construction materials for aircraft, bicycles, boats and cars, to be able to absorb forces in predetermined directions and to be flexible, in particular elastically flexible, in other directions through the special choice of orientation and layering of the carbon fibers. Typically, such carbon materials consist mainly of carbon fibers. The duroplastic matrix essentially only serves to fix the carbon fibers to each other and as an outer protective coating. The present invention makes use of this special designability of carbon material. It enables a very thin design of the back rail, which is practically not bulky and at the same time is compression-resistant in the longitudinal direction, i.e. cranial-caudal. At the same time, sagittal bending elasticity is maintained so that the user can still bend forward or straighten up almost unhindered. This sagittal bending elasticity also ensures that the ventral flat side of the back rail always remains in contact with the back muscles adjacent to the spine, so that the orthosis is supported here. This means that forces introduced into the orthosis are transferred laterally into these back muscles over the entire length of the orthosis. The spine relief is therefore based on a load transfer from a skeletal body structure, namely the spine, to a muscular body structure, namely the back muscles. Although muscular structures can be fatigued, they are much more difficult to damage than skeletal structures. Accordingly, it is always the aim of any training that strengthens the body to build up the muscular structures in such a way that they—instead of skeletal structures—are able to absorb and dissipate external forces. With the orthosis according to the invention, the corresponding force is now introduced directly into the muscular structures, i.e. bypassing the skeletal structures, in particular the spine. In this way, the force of the muscular structures can be used more efficiently, while at the same time reducing the force exerted on the skeletal structures.

The choice of material according to the invention makes it possible to achieve the stability required for force redirection, in particular in the cranial-caudal direction, permanently and simultaneously with flexibility, in particular elastic flexibility, in those directions in which the user requires freedom of movement. This is not or only very inadequately realized in the prior art described above, which either (in the deactivated state) provided complete (non-elastic) flexibility of the back rail or (in the activated state) complete stiffening of the back rail.

As a result, the orthosis according to the invention can be worn permanently without causing the user any discomfort and fulfills its task without consciously switching between different states, which makes the orthosis particularly suitable for use in the context of unpredictable loads, such as during rescue operations or in sports. In addition, the sheet-like carbon material is so thin and flat-fitting that it does not press painfully into the user's back even when back loads, such as oxygen cylinders, are placed on it.

In some embodiments, the back rail has two lateral support regions extending parallel along the longitudinal direction of extension of the back rail and a central region connecting the support regions and arching over the user's spine when in use. The significance of the lateral support areas has already been explained in detail above. By arching over the spine by means of the central area, the spinous processes of the spine are specifically relieved, as their direct contact with the back rail is avoided. In some embodiments, the central region is shaped as a bulge that curves dorsally with respect to the support regions. In other words, the central area forms a ventrally open channel into which the spinous processes can protrude without contact. This shape allows the carbon material of the back rail to have a constant sheet thickness across its width. As a rule, a sheet thickness of a few millimetres, e.g. 1-3 mm, is sufficient to achieve the advantageous properties of the orthosis according to the invention described above. This also ensures effective impact protection for the sensitive spinous processes, which can prove particularly effective in the event of falls.

However, the channel created in the central area can act like a stiffening rib that excessively restricts the sagittal bending elasticity. In a further development of the invention it is therefore envisaged that the central region is divided by means of width-centered transverse slits into a plurality of segments distributed across its length. This does not change the one-piece nature of the back rail and also does not impair the preferred full-surface contact of the lateral areas with the back extensor muscles. However, the flexibility of the back rail in the median sagittal plane is increased. The frontal bending elasticity and torsional elasticity can also be influenced by the special shape and dimensioning of the transverse slits and can therefore be adjusted by the manufacturer to the desired degree in individual cases.

In addition to its sagittal bending elasticity, the back rail is designed to be frontally bending-elastic, with the amount of its frontal modulus of elasticity being at least one order of magnitude greater than that of its sagittal modulus of elasticity. This means that the orthosis according to the invention not only allows the user to bend forward and straighten up, but also—albeit to a limited extent—to bend laterally in a frontal plane. Such movements also are permitted naturally by the spine only to a limited extent and, if used to excess, can quickly lead to serious injury. This natural gradation of mobility is imitated by the orthosis according to the invention in the further development described. This means that lateral bending is permitted, but the user is “reminded” that such movements should ideally be kept to a minimum. Sudden lateral loads to which the user could be exposed in an unforeseen manner are absorbed by the high modulus of elasticity, i.e. the lower flexibility in this direction, so that the orthosis also offers protection against injury.

Similarly, in a further development of the invention, it is provided that the back rail is designed to be torsionally elastic about its direction of longitudinal extension, the amount of its torsional modulus of elasticity being between those of its frontal and its sagittal modulus of elasticity. This also corresponds to an imitation of the natural prioritization of different mobilities of a healthy spine, which is strongly and easily bendable in the (median) sagittal plane, somewhat more limited and more difficult to twist and even more limited and even more difficult to bend in the (median) frontal plane.

The main advantageous properties of the orthosis according to the invention result, as described, from the design of its back rail. However, this can only work in accordance with the invention if it is correctly positioned when in use. In principle, it would be conceivable to fix a separate back rail directly to the user's back, for example by gluing it in place. However, this would significantly limit general acceptance and manageability. Therefore, as already mentioned at the beginning, a backpack-like shoulder carrier system is provided to hold the back rail in position. Conveniently, each of the two carrier elements of the shoulder carrier system is made of a sheet-like carbon material, in particular the same carbon material as the back rail. When in use, each carrier element forms a ring embracing the respectively associated shoulder of the user and, starting from the cranial end of the back rail, being guided back parallel to the user's rib arches to the back rail, to which it is connected in the lower region of the back rail's upper third and/or the upper region of the back rail's middle third, lying flat against the user's shoulder, collarbone and chest muscle. In this embodiment, therefore, no belt system is used. Instead of straps that are flexible in all directions (apart from tensile strength, if necessary), a carbon ring is used that lies flat against the user's body and is elastic in bending everywhere transverse to its respective longitudinal extension and width direction, but is resistant to compression or tension in its longitudinal extension direction and resistant to bending in its width direction. This allows the support elements to be guided in close contact with the contours of the user's body and, as explained above in the context of the lateral support areas of the back rail, serve to introduce external forces into muscular structures, in this case the shoulder and chest muscles. This means that these muscles can also be used to absorb and dissipate the external forces. This leads to a further increase in the efficiency of the spinal support according to the invention.

To ensure good manageability with such a selectively rigid shoulder support system, it is preferable that each carrier element is constructed in two parts, namely an upper half-ring part and a lower half-ring part. The upper half-ring part is formed integrally with the back rail as a single piece of material and is reversibly connectable in the region of its free end, which, when in use lies in the region of the user's chest muscle, to the free end of the lower half-ring part, which is connected to the back rail in a height-adjustable manner. Due to the bending elasticity described above, the upper and lower half-ring parts can be opened wide when separated in such a design, so that the user can comfortably get in. The free ends of the half-ring parts are then joined together in the pectoral muscle area and the support ring is closed so that it then forms the selectively rigid carrier element described above. The one-piece design of the upper half-ring parts with the back rail is favorable in terms of avoiding many individual parts and possibly weak points. The lower half-ring part, on the other hand, should only be connected to the back rail in one piece in exceptional cases. It is generally considered preferable to connect it to the back rail in a reversible, in particular height-adjustable, manner to allow adaptability to users of different heights.

It has proven to be particularly practical if the free ends of the upper lower half-ring part overlap each other when in use and can be reversibly connected to each other, in particular by means of a hook-and-loop fastener. The fixed end of the lower half-ring part can also be connected to the back rail by means of a hook-and-loop fastener, which makes the above-mentioned height adjustability technically easy to implement. The lower half-ring parts of the two support elements are preferably formed in one piece. They preferably merge into one another in the area of their connection with the back rail.

In a further development of the invention, it is further provided that a radially flexible, ventrally open rib bracket made of a sheet-like carbon material, preferably height-adjustable, is connected to the back rail, in particular by means of a hook-and-loop connection, which in the case of use fits flatly around the user's chest from dorsal to ventral. The rib bracket preferably is made of the same sheet-like carbon material as the back rail. Due to its bending elasticity perpendicular to the longitudinal and transverse direction, the rib bracket automatically adapts to the user's chest. Its compressive and tensile strength in the longitudinal direction and its flexural strength in the transverse direction contribute to a stabilizing and centering effect on the back rail. The preferably realized height adjustability of the rib bracket serves the adaptability to different sizes of users.

Straps preferably are fixed to the free ends of the rib bracket. These free ends are spaced apart from one another in the circumferential direction and the straps are reversibly connectable to one another to form a strap connection spanning the distance between the free ends of the rib bracket. This provides additional stability. It is also possible to fit a chest plate between the free ends of the rib bracket, particularly in conjunction with the lower half-ring parts of the shoulder carrier elements, which can serve to protect the solar plexus.

Alternatively or additionally, a radially bending-elastic pelvic bracket may be connected to the back rail. The pelvic bracket is ventrally open, made of a sheet-like carbon material, and embracing, when in use, the user's body flatly from dorsal to ventral along the user's iliac crest. The pelvic bracket is preferably made of the same sheet-like carbon material as the back rail. As explained at the beginning, the central effect of the invention is not to transfer external loads to the user's pelvis; nevertheless, it can be useful to transfer residual forces that could not yet be transferred to muscular structures in the manner described in the invention to the iliac crest. However, this then is subjected to significantly less stress than with orthoses according to the state of the art.

In the context of the pelvic bracket, it may also be provided that straps are fixed to the free ends of the pelvic bracket. The free ends are spaced apart from one another in the circumferential direction and the straps are reversibly connectable to one another to form a strap connection spanning the distance between the free ends of the pelvic bracket. With regard to the effect and advantages of this measure, mutatis mutandis reference is made to the above explanation of the rib bracket.

Further details and advantages of the invention can be seen from the following special description and the drawings.

### BRIEF DESCRIPTION OF THE DRAWINGS

FIG. 1 is a perspective view of the basic framework of a thorax orthosis according to the invention.

FIG. 2 is a frontal view of the orthosis of FIG. 1.

FIG. 3 is a view along the cranial-caudal line of sight of the orthosis of FIG. 1

FIG. 4 shows the orthosis of FIG. 1 with additional comfort and functional features.

### DESCRIPTION OF PREFERRED EMBODIMENTS

Identical reference signs in the figures indicate identical or analogous elements.

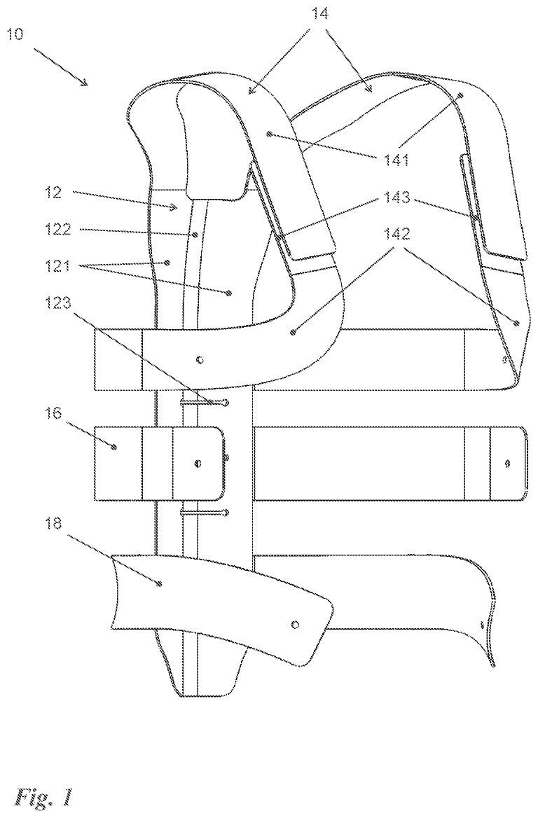

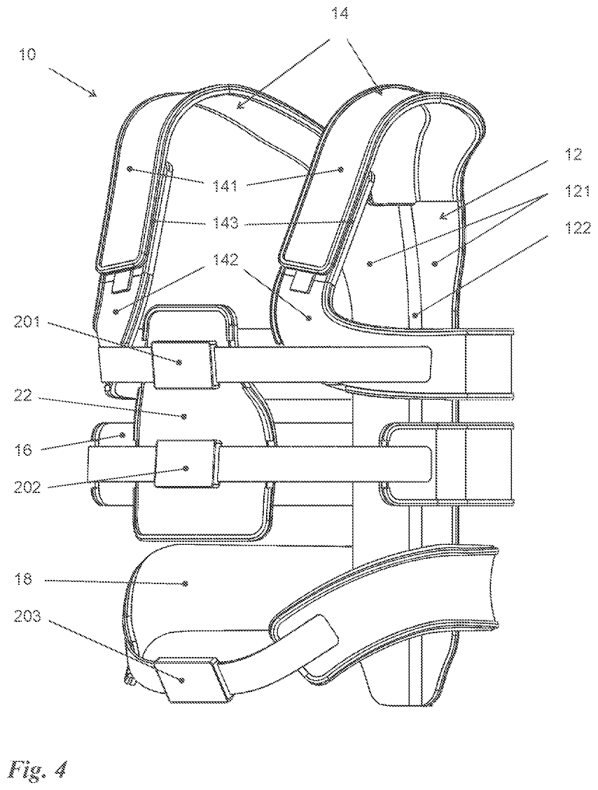

FIGS. 1 to 3 show different views of the basic structure of a preferred embodiment of a thoracic orthosis 10 according to the invention. These figures will first be discussed together. They show the basic framework of the orthosis 10 according to the invention, which is essentially made of sheet-like carbon material and which can be upgraded with additional comfort and functional elements, for example as illustrated in FIG. 4.

An essential component of the orthosis 10 is its back rail 12. In the embodiment shown, the back rail 12 consists of two lateral support areas 121 and a central area 122, which is designed as a dorsally directed bulge and forms a cranial-caudal, ventrally open channel. When in use, the back rail 12 is placed against the back of a user not shown in such a way that its lateral support regions 121 rest on the back extensor muscles adjacent to the user's spine and the spine itself, in particular its spinous processes, is arched over by the channel of the central region 122 without contact. In particular, the channel structure of the central area 122 is clearly recognizable in FIG. 3.

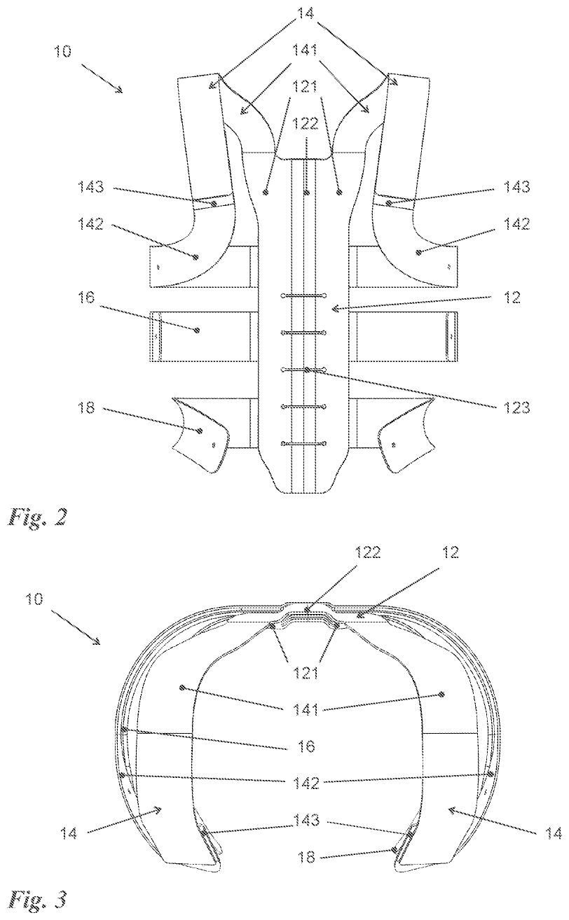

Several transverse slits 123 are arranged along the length of the back rail 12, which span the channel of the central area 123. This counteracts the stiffening effect of the curvature of the central area 122 and increases the sagittal bending elasticity 12. In addition, the transverse slits 123 improve ventilation. In the embodiment shown, the transverse slits 123 end in lateral round holes. This prevents the ends of the transverse slits from tearing out even in the event of frequent, sharp bends. In addition, this shape, together with a slit width that can be selected depending on the individual case, ensures torsional and frontal elasticity that can be adjusted by the manufacturer, i.e. the back rail 12 can be slightly bent and twisted frontally, i.e. in FIG. 2 in the plane of the drawing, without the opposite edges of the slits colliding with each other during movement and possibly trapping pieces of clothing or even skin.

The caudal end of the back rail 12 is tapered to adapt to the natural anatomy in the user's lumbar region, whereby care has been taken to ensure that the lines are rounded and avoid any corners.

Two upper half-ring parts 141 of two carrier elements 14 are molded onto the cranial ends of the back rail 12. They are made of the same sheet-like carbon material as the back rail 12. When in use, they run over the user's shoulders and end with their free ends approximately in the area of the user's chest muscles. Here they are reversibly connected, in particular by means of a hook-and-loop fastener 143, to the free ends of lower half-ring parts 142, which extend from the chest muscle region parallel to the rib arches of the user from ventral to dorsal to the back rail 12. There they are preferably reversibly fixed, in particular by means of a further hook-and-loop fastener on the dorsal outer side of the back rail 12. In the preferred embodiment shown, the two lower half-ring parts 142 are formed in one piece with one another and merge into one another in the same material, particularly in the area where they are fixed to the back rail 12. The reversibility of their attachment to the back rail 12 allows height adjustment and thus size adjustment of the arm cut-outs of the support elements 14, so that the orthosis 12 can be adapted within certain limits to users of different heights. It is therefore possible to limit production to a small number of basic sizes and to enable an individually optimized fit by fine-tuning on the user's side. Due to the material properties of sheet-like carbon material discussed in detail in the general part of the description, the upper half-ring parts can be elastically bent upwards without great effort when the hook-and-loop fastener 143 is open. The lower half-ring parts 142, on the other hand, can be bent open outwards to the side. This makes it easy for the user to get into the orthosis 12.

After fixing the free ends of the upper and lower half-ring parts 141, 142 by means of the hook-and-loop fasteners 143, however, the half-ring parts 141, 142 prevent the respectively other half-ring parts 142, 141 from bending, so that an essentially rigid support ring is created overall, which is supported directly on the user's shoulder and chest muscles. Together with the back rail 12, the support elements 14 therefore ensure that external forces are transferred away from the user's spine to a wide range of muscular structures. These can absorb the forces together without overloading individual muscles. This leads to a particularly efficient relief of the spinal column by the orthosis according to the invention and thus to a significant increase in the user's performance.

In the embodiment shown, a rib bracket 16 is arranged caudal to the lower half-ring parts 142 of the support elements 14, which, similar to the lower half-ring elements 142, are preferably reversibly fixed to the dorsal outer side of the back rail 12, in particular by means of a hook-and-loop fastener. They are also preferably made of the same carbon material as the back rail 12 and support elements 14 and adapt flexibly to the user's upper body in the area of the lower rib arches. They can be bent outwards to the side for access. They are primarily used to center the back rail 12 in its central area.

Near the caudal end of the back rail 12, a pelvic bracket 18 is also provided in the embodiment shown, which is preferably also reversibly fixed to the dorsal outer side of the back bracket 12, in particular also by means of a hook-and-loop fastener. The pelvic bracket 18 embraces the user's body from dorsal to ventral along the iliac crest. Unlike the rib bracket 16, its slightly tilted bend, which is particularly easy to recognize in FIGS. 1 and 2, allows vertical load support on the iliac crest. However, as explained in detail in the general part of the description, this is only a secondary effect. The primary purpose of the pelvic bracket 18 is to center the back rail 12 in its caudal end region.

The pure carbon frame (possibly with connecting devices), in particular hook-and-loop fasteners, as shown in FIGS. 1 to 3, is basically sufficient to achieve the effects according to the invention explained in the general part of the description. However, it is easily possible, for example for reasons of comfort, to provide additional equipment. For example, the carbon frame can be completely or locally lined or coated with padding elements, such as gel padding or neoprene layers. A correspondingly upgraded orthosis 12 is shown in FIG. 4.

In addition to said padding, the orthosis 12 of FIG. 4 has additional strap connections 201, 202, 203, which connect the opposing areas or ends of the lower half-ring parts 142 (strap connection 201), rib bracket 16 (strap connection 202) and pelvic bracket (strap connection 203) to one another. This makes it possible to achieve a more stable fixation of the back rail 12, which can be pulled closer to the user's spine in particular by the strap connections 201, 202, 203.

In the embodiment shown, a chest plate 22 is fixed in the area of the fasteners of the two upper strap connections 201, 202. This is preferably also made of a sheet-like carbon material, in particular the same carbon material as the back rail 12, and can be provided with padding, for example coated, on its inner side.

Such a chest plate 22 provides effective impact protection for the user's solar plexus. It also provides an additional contact surface to the user's muscular structures, which can be used to absorb the external forces transferred via the orthosis 12. Of course, the embodiments discussed in the specific description and shown in the figures are only illustrative examples of the present invention. In the light of the present disclosure, the skilled person is provided with a wide range of possible variations.

### List of Reference Symbols

10 orthesis

12 back rail

121 lateral support area of 12

122 central area of 12

123 transverse slit

14 carrier element

141 upper half ring part of 14

142 lower half ring part of 14

143 hook-and-loop fastener

16 rib bracket

18 pelvic bracket

201 strap connection

202 strap connection

203 strap connection

22 breast plate

1. A thoracic orthosis (10) for relieving a human user's spine when required, comprising
a back rail (12) resting, when in use, with a ventral flat side against back extensor muscles adjacent to both sides of the user's spine in a region of the thoracic and at least the upper lumbar vertebrae of the user, and
a shoulder carrier system connected to the back rail (12) and having two carrier elements (14) reaching around the user's shoulders in a backpack-style when in use, the back rail (12) being made in one piece from a sheet-like carbon material that is compression-resistant in its direction of longitudinal extension and is sagittally bending-elastic.

2. The thoracic orthosis (10) of claim 1, wherein the back rail (12) has two lateral support regions (121) extending parallel along the longitudinal direction of extension of the back rail (12) and a central region (122) connecting the support regions (121) and arching over the user's spine when in use.

3. The thoracic orthosis (10) of claim 2, wherein the central region (122) is shaped as a bulge that curves dorsally with respect to the support regions.

4. The thoracic orthosis (10) of claim 3, wherein the carbon material of the back rail (12) has a constant sheet thickness across its width.

5. The thoracic orthosis (10) of claim 2, wherein the central region (122) is divided by means of width-centered transverse slits (123) into a plurality of segments distributed across its length.

6. The thoracic orthosis (10) of claim 1, wherein the back rail (12) is designed to be frontally bending-elastic, the amount of its frontal modulus of elasticity being at least one order of magnitude greater than that of its sagittal modulus of elasticity.

7. The thoracic orthosis (10) of claim 6, wherein the back rail (12) is torsionally elastic about its direction of longitudinal extension, the amount of its torsional modulus of elasticity being between those of its frontal and its sagittal modulus of elasticity.

8. The thoracic orthosis (10) of claim 1, wherein each carrier element (14) of the shoulder carrier system is made from a sheet-like carbon material and, when in use, forms a ring embracing the respectively associated shoulder of the user and, starting from the cranial end of the back rail (12), being guided back parallel to the user's rib arches to the back rail (12) to which it is connected in the lower region of the back rail's (12) upper third and/or the upper region of the back rail's (12) middle third, lying flat against the user's shoulder, collarbone and chest muscle.

9. The thoracic orthosis (10) of claim 8, wherein the carrier element (14) is constructed in two parts, namely an upper half-ring part (141) and a lower half-ring part (142), the upper half-ring part (141) being formed integrally with the back rail (12) as a single piece of material and being reversibly connectable in the region of its free end, which, when in use lies in the region of the user's chest muscle, to the free end of the lower half-ring part (142), which is connected to the back rail (12) in a height-adjustable manner.

10. The thoracic orthosis (10) of claim 9, wherein the free ends of the upper half-ring part (141) and of the lower half-ring part (142) overlap one another when in use and can be connected to one another reversibly.

11. The thoracic orthosis (10) of claim 9, wherein the lower half-ring part (142) is connected to the back rail (12) by a hook-and-loop connection.

12. The thoracic orthosis (10) of claim 1, further comprising a radially bending-elastic rib bracket (16) being open ventrally and, when in use, embracing the user's thorax flatly from dorsal to ventral, and being made of a sheet-like carbon material is connected to the back rail (12).

13. The thoracic orthosis (10) of claim 12, further comprising straps fixed to the ends of the rib bracket (16), said free ends being spaced apart from one another in the circumferential direction and said straps being reversibly connectable to one another to form a strap connection (202) spanning the distance between the free ends of the rib bracket (16).

14. The thoracic orthosis (10) of claim 1, further comprising a radially bending-elastic pelvic bracket (18) connected to the back rail (12), said pelvic bracket (18) being ventrally open, made of a sheet-like carbon material, and embracing, when in use, the user's body flatly from dorsal to ventral along the user's iliac crest.

15. The thoracic orthosis (10) of claim 14, further comprising straps fixed to the free ends of the pelvic bracket (16), said free ends being spaced apart from one another in the circumferential direction and said straps being reversibly connectable to one another to form a strap connection (203) spanning the distance between the free ends of the pelvic bracket (16).
